(19) United States
(12) Patent Application Publication
(10) Pub. No.: US 2008/0132577 A1
(43) Pub. Date: Jun. 5, 2008

(54) Use of agomelatine in obtaining medicaments intended for the treatment of smith-magenis syndrome

(75) Inventors: Elisabeth Mocaer, Neuilly Sur Seine (FR); Agnes Fabiano, Verona (IT)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, COURBEVOIE CEDEX (FR)

(21) Appl. No.: 11/986,501

(22) Filed: Nov. 21, 2007

(30) Foreign Application Priority Data
Nov. 24, 2006 (FR) 06.10296

(51) Int. Cl. A61K 31/164; A61P 43/00

(52) U.S. Cl. 514/630

(57) ABSTRACT

The present invention relates to the use of agomelatine, or N-[2-(7-methoxy-1-naphthyl)ethyl]acetamide, in obtaining medicaments intended for the treatment of Smith-Magenis syndrome.

The present invention relates to the use of agomelatine, or N-[2-(7-methoxy-1-naphthyl)ethyl]acetamide of formula (I):

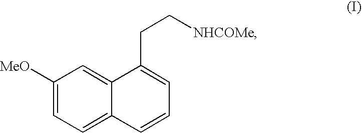

and also its hydrates, crystalline forms and addition salts with a pharmaceutically acceptable acid or base, in obtaining medicaments intended for the treatment of Smith-Magenis syndrome.

Agomelatine, or N-[2-(7-methoxy-1-naphthyl)ethyl]acetamide, has the double characteristic of being, on the one hand, an agonist of receptors of the melatoninergic system and, on the other hand, an antagonist of the 5-HT_{2C} receptor. These properties provide it with activity in the central nervous system and, more especially, in the treatment of major depression, seasonal affective disorder, sleep disorders, cardiovascular pathologies, pathologies of the digestive system, insomnia and fatigue due to jet-lag, appetite disorders and obesity.

Agomelatine, its preparation and its use in therapeutics have been described in European Patent Specifications EP 0 447 285 and EP 1 564 202.

The Applicant has now found that agomelatine or N-[2-(7-methoxy-1-naphthyl)ethyl]-acetamide—and also its hydrates, crystalline forms and addition salts with a pharmaceutically acceptable acid or base—has valuable properties allowing its use in the treatment of Smith-Magenis syndrome.

Described by Ann Smith et al. in 1982 (Smith A. C. M. et al., 1986, Am. J. Med. Genet., 24, 393-414), Smith-Magenis syndrome (SMS) is a rare genetic disorder due to a chromosomal microdeletion. This especially serious disorder causes, in children, the appearance of a dysmorphic syndrome, mental retardation, especially relating to the acquisition of language, hyperactivity with attention deficit, and autoaggression associated with serious behaviour and sleep disorders (Smith, A. C. M. et al., 1998, Am. J. Med. Genet., 81, 186-191).

It is necessary to add to these elements the distress of the child's family circle facing the disorder, who not only have to accept the handicap but are in addition exposed to extreme living conditions (constantly disturbed nights, increased vigilance at all times, the child's aggression having to be controlled, etc.), which in most cases leads to total disruption of the family unit.

The incidence is one in 25,000 live births. The diagnosis is based on the clinical signs and is confirmed by demonstration of the deletion from chromosome 17 by means of a high-resolution karyotype. An inverted circadian rhythm of melatonin has recently been demonstrated and may well be the cause of the sleep disorders and behaviour disorders (De Leersnyder H., 2006 Trends in Endocrinology and Metabolism, 17(7), 29-298).

Although there is no truly satisfactory and recognised treatment for SMS, the drugs currently used the most are neuroleptics, hypnotics, psychostimulants, antidepressants, antipsychotics and carbamazepine for controlling the behaviour disorders. These treatments are the cause of numerous secondary effects such as gastrointestinal disorders, weight gain, dyslipidaemias, sexual dysfunction, tardive dyskinesias and a cardiovascular impact (Richelson E. et al., 1999, J. Clin. Psychiatry, 60(10), 5-14; Trenton A. J. et al., 2003, CNS Drugs, 17 (5), 307-324; Freedman R. et al., 2003, New England Journal of Medicine, 343, 1738-1749). These treatments moreover do not have any activity in respect of the desynchronisation of melatonin secretion, which is the cause of the major sleep disorders and certain behaviour disorders whose impact is especially disruptive for the child and the child's family.

Currently, the strategy for treating the desynchronisation of melatonin secretion is a morning administration of beta-adrenergic antagonists (acebutolol, propranolol) in order to block the endogenous secretion of melatonin, associated with an evening administration of exogenous melatonin (De Leersnyder H. et al., 2003, J. Med. Genet., 40, 74-78). However, the melatonin has no activity in respect of the behaviour disorders.

Accordingly, making available new treatments for this orphan pathology of children is of major interest. In particular, perfecting a treatment allowing simultaneous alleviation of the major sleep disorders and of the behaviour disorders would allow the child and the child's family circle to regain a more reasonable quality of life.

The Applicant has now found that agomelatine, by virtue of its pharmacological characteristics, is especially suitable in this indication. In fact, agomelatine makes it possible to act simultaneously on the behaviour disorders and on the resynchronisation of disturbed circadian rhythms. Agomelatine moreover has no drug interactions and has an optimum acceptability profile: more than 4000 patients have been exposed to agomelatine in the course of the clinical trials that have been carried out and it is has been possible to observe excellent clinical and biological tolerability.

The invention accordingly relates to the use of agomelatine, and also its hydrates, crystalline forms and addition salts with a pharmaceutically acceptable acid or base, in obtaining pharmaceutical compositions intended for the treatment of Smith-Magenis syndrome.

The invention relates especially to the use of agomelatine obtained in crystalline form II, described in Patent Application EP 1 564 202, in obtaining pharmaceutical compositions intended for the treatment of Smith-Magenis syndrome.

The pharmaceutical compositions will be presented in forms suitable for administration by the oral, parenteral, transcutaneous, nasal, rectal or perlingual route, and especially in the form of injectable preparations, tablets, sublingual tablets, glossettes, gelatin capsules, capsules, lozenges, suppositories, creams, ointments, dermal gels etc.

Besides agomelatine, the pharmaceutical compositions according to the invention comprise one or more excipients or carriers selected from diluents, lubricants, binders, disintegration agents, absorbents, colourants, sweeteners etc.

By way of non-limiting example there may be mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: aluminium and magnesium silicate, starch, gelatin, tragacanth, methylcellulose, sodium carboxymethylcellulose and polyvinylpyrrolidone,
as disintegrants: agar, alginic acid and its sodium salt, effervescent mixtures.

The useful dosage varies according to the age and weight of the patient, the administration route, the nature of the disorder and any associated treatments and ranges from 1 mg to 50 mg of agomelatine per 24 hours.

Preferably, the daily dose of agomelatine will be 25 mg per day, with the possibility of increasing to 50 mg per day.

### Pharmaceutical Composition:

Formula for the preparation of 1000 tablets each containing 25 mg of active ingredient:

| | | |
|---|---|---|
| N-[2-(7-methoxy-1-naphthyl)ethyl]acetamide | 25 | g |
| Lactose monohydrate | 62 | g |
| Magnesium stearate | 1.3 | g |
| Povidone | 9 | g |
| Anhydrous colloidal silica | 0.3 | g |
| Cellulose sodium glycolate | 30 | g |
| Stearic acid | 2.6 | g |

### Clinical Study:

An exploratory phase II study was carried out in children with Smith-Magenis syndrome. Agomelatine 1-5 mg/kg was co-administered with acebutolol 10 mg/kg, a PI adrenergic antagonist. The main analysis criteria were actigraphy parameters recorded over five periods of 30 consecutive days, after 30 days of treatment, and 3, 5 and 15 months of treatment, and also the Achenbach questionnaire, allowing the behaviour disorders to be assessed.

The results obtained show that with agomelatine there is a reduction in the frequency and duration of night waking, which is accompanied by a reduction in the duration of daytime naps. Major clinical improvement was found by a specialist in this pathology for those children who were treated with agomelatine: for the first time a calm, deep sleep, less-broken nights and less-early waking in the morning were recorded. Real progress in relation to behaviour was also observed. These major effects were carried over to the families, who, following the highly positive consequences of the treatment on family life, requested continuation of the treatment on a compassionate basis.

1- A method for treating a living animal body, including a human, afflicted with Smith-Magenis syndrome comprising the step of administering to the living animal body, including a human, an amount of agomelatine, or N-[2-(7-methoxy-1-naphthyl)ethyl]acetamide, or a hydrate, crystalline form or addition salt thereof with a pharmaceutically acceptable acid or base, which is effective for treatment of Smith-Magenis syndrome.

2- The method of claim 1, wherein the agomelatine is in crystalline form II.

3- A pharmaceutical composition comprising agomelatine, or a hydrate, crystalline form or addition salt thereof with a pharmaceutically acceptable acid or base, alone or in combination with one or more pharmaceutically acceptable excipients.

4- The pharmaceutical composition of claim 3, wherein the agomelatine is in crystalline form II.
